(19) United States
(12) Patent Application Publication
(10) Pub. No.: US 2021/0174945 A1
(43) Pub. Date: Jun. 10, 2021

(54) METHODS AND SYSTEMS FOR FILLING OPEN APPOINTMENTS

(71) Applicant: SureShow Inc., Kansas City, KS (US)

(72) Inventors: Shelley Cooper, Kansas City, MO (US); Moe Hamid, Shawnee, KS (US)

(73) Assignee: SureShow Inc., Kansas City, KS (US)

(21) Appl. No.: 17/114,002

(22) Filed: Dec. 7, 2020

Related U.S. Application Data
(60) Provisional application No. 62/945,120, filed on Dec. 7, 2019.

(51) Int. Cl. G16H 40/20; G06Q 10/10; G16H 40/67; G16H 70/60; G16H 10/60; G06F 16/2457; G06F 16/28

(52) U.S. Cl. CPC G16H 40/20; G06Q 10/1095; G16H 40/67; G06F 16/285; G16H 10/60; G06F 16/24578; G16H 70/60

(57) ABSTRACT

Methods and systems for filling open appointments with a telehealth encounter. A method comprises determining a selected classification code from one or more classification codes associated with one or more clients in a client database associated with a service provider; detecting an event indicative of a change in calendar data, the change in calendar data comprising an opening of a time slot in a schedule of the service provider; determining that the opening should be filled; generating a list of one or more clients associated with the selected classification code from the client database; sending a signal representative of a notification regarding the opening of the time slot to one or more devices associated with the one or more clients associated with the selected classification code; and connecting the device associated with the client with a device associated with the service provider.

### CROSS-REFERENCE TO RELATED APPLICATIONS

This non-provisional patent application claims priority benefit, with regard to all common subject matter, of commonly assigned U.S. provisional patent application Ser. No. 62/945,120, filed Dec. 7, 2019, and entitled “SYSTEM FOR FILLING OPEN APPOINTMENTS” (“the '120 application”). The '120 application is hereby incorporated by reference in its entirety into the present non-provisional patent application.

### BACKGROUND

Scheduling systems are often configured to send reminders of scheduled appointments. Depending on the frequency of appointments, the reminder timeframes are usually anywhere from at least two weeks out to the day before an appointment. In some systems, the reminders involve having a receptionist call to remind clients, which is labor-intensive, often ineffective, and expensive. Further, such systems do not have a sizeable impact on decreasing no-shows or last-minute appointment cancellations, and they do not address the lack of generated revenue when the appointment is left open. To avoid such lost revenue, firms often employ cancellation policies that are burdensome and cost-prohibitive to financially insecure clients.

This background discussion is intended to provide information related to the present invention which is not necessarily prior art.

### BRIEF SUMMARY

The present invention solves the above-described problems and other problems by providing methods and systems for filling appointments that avoid lost revenue due to no-shows and last-minute cancellations.

A computer-implemented method according to an embodiment of the present invention comprises: determining, via one or more processors, a selected classification code of one or more classification codes associated with one or more clients in a client database associated with a service provider; detecting, via the one or more processors, an event indicative of a change in calendar data, the change in calendar data comprising an opening of a time slot in a schedule of the service provider; determining, via the one or more processors, that the opening of the time slot should be filled; generating, via the one or more processors, a list of one or more clients associated with the selected classification code from the client database; and sending, via the one or more processors, a signal representative of a notification regarding the opening of the time slot to one or more devices associated with the one or more clients associated with the selected classification code.

A system constructed according to an embodiment of the present invention comprises a client database, a calendar database, and a first computing device. The client database is configured to store electronic medical records for clients. The electronic medical records include client identification data, classification codes associated with client identification data, and contact data associated with client identification data. The calendar database is configured to store calendar data associated with a service provider. The calendar data comprises time slots assigned to clients representative of appointments.

The first computing device is in communication with the client database and the calendar database and is configured to detect an event indicative of a change in the calendar data, the change in the calendar data comprising an opening of a time slot of the service provider; generate a list of one or more classification codes associated with one or more clients in the client database; receive a signal representative of a selected classification code; generate a list of one or more clients associated with the selected classification code from the client database; and send a signal representative of a notification regarding the opening of the time slot using the contact data associated with the one or more clients associated with the selected classification code.

A method according to another embodiment of the present invention comprises receiving, via one or more processors, a signal representative of a user input comprising a request for a joint appointment with a client and a second type of service provider; sending, via the one or more processors, a signal representative of a request for calendar data associated with the first type of service provider and calendar data associated with the second type of service provider to a server; receiving, via the one or more processors, a signal representative of the calendar data associated with the first type of service provider and calendar data associated with the second type of service provider from the server; determining, via the one or more processors, a shared available time slot for the first type of service provider and the second type of service provider based on the calendar data associated with the first type of service provider and calendar data associated with the second type of service provider; and sending, via the one or more processors, a signal representative of a notification of the shared available time slot to a device associated with the client.

Advantages of these and other embodiments will become more apparent to those skilled in the art from the following description of the exemplary embodiments which have been shown and described by way of illustration. As will be realized, the present embodiments described herein may be capable of other and different embodiments, and their details are capable of modification in various respects. Accordingly, the drawings and description are to be regarded as illustrative in nature and not as restrictive.

### BRIEF DESCRIPTION OF THE DRAWINGS

The Figures described below depict various aspects of systems and methods disclosed therein. It should be understood that each Figure depicts an embodiment of a particular aspect of the disclosed systems and methods, and that each of the Figures is intended to accord with a possible embodiment thereof. Further, wherever possible, the following description refers to the reference numerals included in the following Figures, in which features depicted in multiple Figures are designated with consistent reference numerals.

FIG. 1 illustrates various components, in block schematic form, of an exemplary system for filling an open appointment;

FIG. 2 illustrates various components of an exemplary client database shown in block schematic form that may be used with the system of FIG. 1;

FIG. 3 illustrates various components of an exemplary calendar database shown in block schematic form that may be used with the system of FIG. 1

FIG. 4 illustrates various components of an exemplary computing device shown in block schematic form that may be used with the system of FIG. 1;

FIG. 5 illustrates various components of an exemplary computing device shown in block schematic form that may be used with the system of FIG. 1;

FIG. 6 illustrates various components of an exemplary client device shown in block schematic form that may be used with the system of FIGS. 1; and

FIG. 7 is a flowchart illustrating at least a portion of the steps for filling an appointment according to an embodiment of the present invention.

The Figures depict exemplary embodiments for purposes of illustration only. One skilled in the art will readily recognize from the following discussion that alternative embodiments of the systems and methods illustrated herein may be employed without departing from the principles of the invention described herein.

### DETAILED DESCRIPTION

The following detailed description of the invention references the accompanying drawings that illustrate specific embodiments in which the invention can be practiced. The embodiments are intended to describe aspects of the invention in sufficient detail to enable those skilled in the art to practice the invention. Other embodiments can be utilized and changes can be made without departing from the scope of the present invention. The following detailed description is, therefore, not to be taken in a limiting sense. The scope of the present invention is defined only by the appended claims, along with the full scope of equivalents to which such claims are entitled.

In this description, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the present technology can include a variety of combinations and/or integrations of the embodiments described herein.

Methods and systems according to embodiments of the present invention enable rapid and dynamic filling of time slots for a service provider (such as a healthcare provider, a Federally Qualified Health Center (FQHC), or the like) when an opening occurs. Embodiments of the present invention rapidly fill openings of any time slot in a schedule, such as an opening occurring in real time/presently, or an opening that is days ahead on a schedule. The openings can be filled with virtual or physical appointments.

For example, when a client, such as a healthcare patient or the like, does not participate in a scheduled appointment (e.g., is a “no-show” for a face-to-face appointment or a virtual appointment) and/or cancels last minute, an opening event occurs. As another example, an opening event may occur when a service provider adds a new slot to its schedule at a specified time. Openings may also occur when cancellations are made for future appointments (e.g., days ahead in a schedule).

Embodiments of the present invention enable one or more substitute client(s) to be given the option of seeing the service provider at least partly within the time window of the opening. In some embodiments, the substitute client(s) may not be already associated with or a client of the service provider, but may instead be a potential client represented in a database of clients interested in receiving opening notifications from the service provider. Unless otherwise specified, “client” as used herein includes potential clients.

Embodiments of the present invention enable the service provider to generate revenue even when a client is a no-show and/or a cancellation, or when an opening otherwise appears in the schedule. One of ordinary skill will appreciate that a provider may operate within any industry and may be part of any organization that schedules appointments without departing form the scope of the present invention.

Embodiments of the present invention enable a client who has expressed interest in receiving notifications regarding an opening of one or more providers to receive such notifications. The embodiments allow a client to enroll for such notifications via her or his provider, to create an account associated with the client, to sign up via a website, and/or to download a mobile application. Embodiments of the present invention allow a client to preselect a timeframe or range of time during enrollment/registration and/or via preference settings. The timeframe may correspond to periods of time in which a client is interested in receiving notifications about provider availability. The timeframe may include periods of availability. For instance, a first timeframe of availability may be Monday morning(s) between eight (8) and eleven (11) AM, and a second timeframe of availability may be Tuesday morning(s) between eight (8) and ten (10) AM. Embodiments of the present invention also allow the client to pre-select one or more provider(s). However, it should be noted that embodiments of the present invention may proactively identify clients for inclusion in a notification process, without prior enrollment activities having been undertaken by the client, without departing from the spirit of the present invention.

Embodiments of the invention enable automatic and/or manual detection of an opening, which allows a provider to immediately determine whether to fill the opening. When an opening occurs, such as an opening for an appointment on the same day, embodiments generate a list of clients to contact for rapidly filling the opening. Embodiments also generate a contact list from a list of clients. The contact list may comprise, for example, those clients who are willing to be contacted at or with respect to the time of the opening. Embodiments also allow the contact list to be ordered according to certain criteria. For instance, a list may be ordered with preference for early adopters, with preference for clients or members that have received fewer notifications by the system and/or have not received a notification recently, according to an algorithm that takes into account a combination of such factors, or the like. The contact list may also or alternatively be ordered based on provider preference(s) or the like. The list may be generated and/or ordered any number of ways without departing from the scope of the present invention.

For example, some embodiments allow the providers to utilize an International Classification of Diseases (ICD) codes, e.g., ICD10 codes, to generate the list. For example, embodiments enable a provider to opt-in all diabetic patients into the list (endocrine, nutritional, and metabolic diseases (E00-E89)), or to opt-out all patients with the nervous system (diseases of the nervous system (G00-G99)). Some embodiments allow a provider to utilize a first letter of a code in addition to a first number and a second number to generate the list.

Some embodiments allow automatic transmission of messages to one or more clients without the need for provider direction. For example, some embodiments allow cycling through a group of clients and, upon receiving an indication of interest in participating in a session with the provider, matching the responding client to an available provider.

Some embodiments also allow a provider to match with one or more other providers and a client. Some embodiments enable a provider to schedule a telemedicine visit with another provider and a single client at the same time. For example, embodiments enable a general practitioner and a specialist to schedule a session with a patient. Such sessions enhance the synergy between multiple experts in determining a treatment plan for the patient. This also allows providers and patients to participate in concurrent research studies that will benefit other patients. Embodiments also allow one or more providers to match with a client and one or more third parties, such as a caregiver.

Embodiments enable notifying one or more clients when an opening occurs and a course of action for filling the opening is selected or otherwise defined. Embodiments enable cycling through the list of clients on a continuous and/or rolling basis, and providing notifications of opening(s) or availability to the clients on the list according to the ordering of the list unless and until one of the clients chooses to take advantage of the opening/availability.

Embodiments may enable the restriction of notifications to only clients having availabilities that overlap the time of the opening. Embodiments of the present invention enable sending an alert to one or more such client(s) through any of a variety of means of communication, such as a mobile application downloaded onto a mobile device from a mobile application store, a mobile text message, an email, an automated calling/messaging system, or the like. Embodiments enable particular messages, such as scripts that read “Doctor is available now. Catch a chance to schedule an appointment with your doctor within 1 minute.” Embodiments that include automated messaging enable notifications that comprise a message to a mobile device that requests a patient/client to “press 1” if the patient/client wants to accept the substitute session or appointment, or to “press 2” if not. Embodiments also allow notifications to include or to reference links or other means for participating in a substitute session or appointment (e.g., virtual appointment) should the client so choose.

When a client declines an opening, embodiments allow for automatically skipping the client until the next instance of a pre-defined timeframe. Embodiments also allow a provider to refrain from sending any additional notifications regarding an opening during a present instance of a client availability timeframe (i.e., to “skip” that client on the rolling list until the next occurrence of availability during that timeframe).

In one or more embodiments, software of the service provider may automatically adjust availability timeframes defined by the client based on acceptance or rejection of one or more openings. For example, the software may be pre-configured to omit the client from a client list generated for an opening that would otherwise match with the client's preferred availability timeframes wherever the client has previously declined one or more opening(s) during that timeframe. For instance, if a service provider has provided notifications for openings on Tuesday mornings on two prior occasions, and each time the client has rejected the openings, the first computing device may determine that a threshold number of rejections has been received by the client, and may omit the client from receiving further opening notifications relating to Tuesday morning openings. Such adjustments to client availability may be permanent, or may sunset according to pre-determined measures (e.g., the adjustment may be reversed after a pre-determined time period has elapsed, such as four (4) weeks after the first rejection). The service provider may manually reverse any such adjustment.

Embodiments also allow a provider to flag a client as opting out in a client database, and allow a client to opt-in and/or opt-out on his or her own initiative.

Embodiments allow a client to indicate acceptance of an opening. When the opening is near-term (i.e., within a pre-determined future timeframe), embodiments may enable rapid gathering of client data, such as via follow-up questions triggered by acceptance, via selections of options for participation, and/or via other invitations for the client to provide such information. This may also include the client authenticating his or her identity through signing into a previously established account or through other means. For example, embodiments enable the client to enter a provider organization's private portal as a means for authentication, client data submission and/or for participating in an appointment. This may also include gathering of billing information, such as insurance and other payment details.

Embodiments enable the provider to access client data before, during, and/or after the appointment. The client data may include medical, dental, and/or other records.

Once the one or more providers and the client are ready to participate in a session at least partly overlapping with an opening, embodiments provide for transmission of a message (e.g., in the form of audio or text) to the client directing her or him to join or arrive and wait for the provider to join the session (e.g., via physical attendance or via a telehealth encounter, such as a phone call, a teleconference, video teleconference, etc.). Embodiments also provide a notification for the provider and a link or other means to join a remote session. The appointment during the opening may be scheduled upon determination of the substitute patient/client's desire to participate, or at a future time acceptable to the provider and the patient/client. Any delay preceding the appointment may be pre-determined, such as where a provider selects an option during enrollment that automatically provides a five-minute buffer between confirmation by the client and the start of a substitute session. Embodiments also enable appointments to be oriented according to pre-determined selections made by the client during enrollment and/or selections made in real-time in response to the notification with respect to when the appointment should begin.

Embodiments may restrict clients from accepting openings beginning within a pre-defined future timeframe (e.g., within five (5) minutes) if the opening requires a pre-defined type of participation from the client that is unlikely to be achieved given such short notice. For example, embodiments may only permit acceptance of such “immediate” openings following responses to corresponding notifications if the openings do not require physical proximity of the client and the provider (e.g., an immediately following substitute session may only be available via remote communication means such as where the substitute session is to be conducted via teleconference or the like).

Embodiments allow for the collection of client data before, during, and after the appointment that fills the opening. For example, the client data may comprise value-based care data on patients for Healthcare Effectiveness Data and Information Set (HEDIS) measures to be submitted to the state. Embodiments also enable client data to be updated, such as electronic medical record(s). Embodiments also enable prescriptions to be written, further treatment plan(s) to be administered, and billing for the appointment.

### Exemplary System

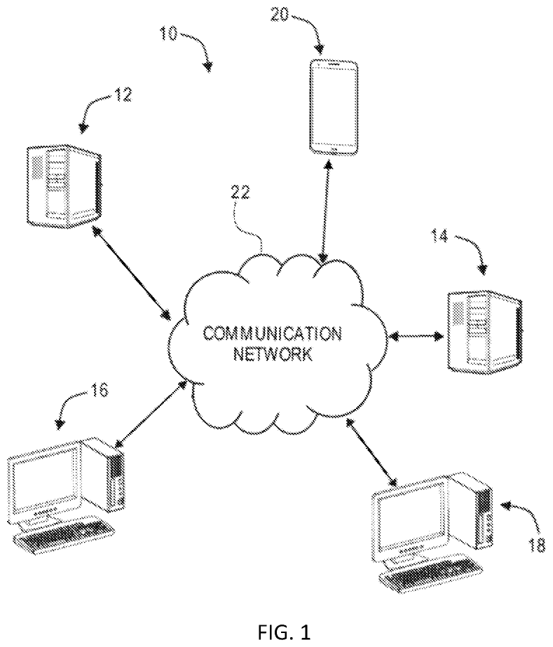

FIG. 1 depicts an exemplary environment 10 for filling openings according to embodiments of the present invention. The environment 10 may include a client database 12, a calendar database 14, a first computing device 16 associated with a first provider, a second computing device 18 associated with a second provider, one or more devices 20 associated with a client, and a communication network 22.

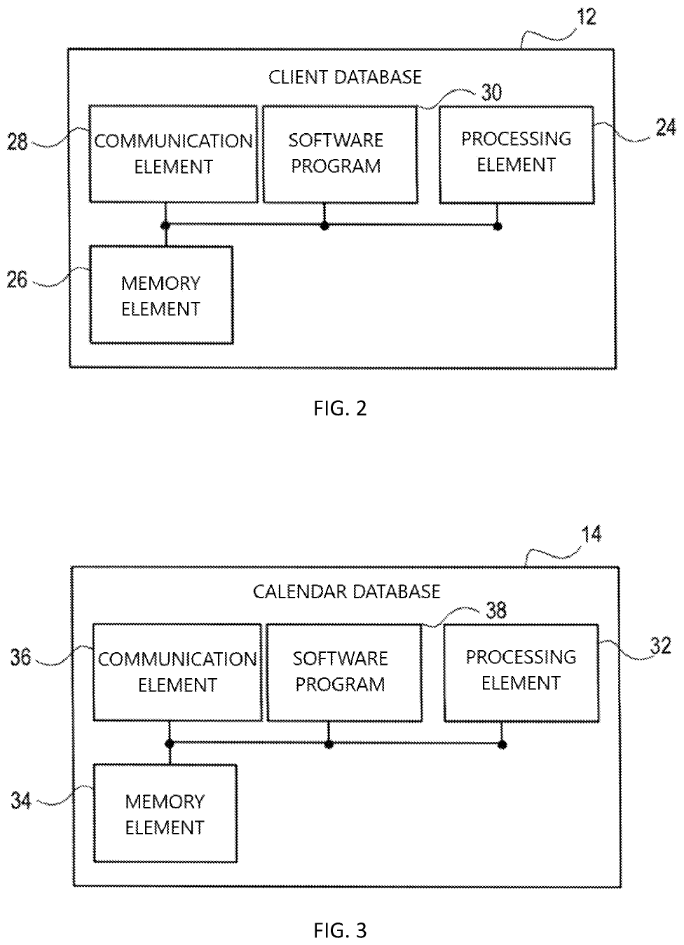

The client database 12 may generally be used for storing client data, such as client health records, billing information, contact information, and information related thereto. The client database 12 may be one or more servers devoted to database applications, such as a database server, and may include software applications stored thereon configured to categorize, organize, and/or store data. For example, the client database 12 may be configured to run a Redox® engine or the like. The client database 12 may include a processing element 24, a memory element 26, a communication element 28, and a software program 30, as shown in FIG. 2. For example, one or more client database 12 may utilize electronic health records (EHR) software as its software program 30, which may be configured for receiving, providing, modifying, and storing EHR. The client database 12 may be a cloud database accessible via the communication network 22 or a local database at the physical location of a service provider. In some embodiments, the database 12 may be integrated with one or more of the computing devices 16, 18 of the service providers.

The calendar database 14 may generally be used for storing scheduling for the first service provider, such as times and dates of appointments, clients assigned to the appointments, practitioners associated with the first service provider, and information related thereto. The calendar database 14 may be one or more servers devoted to database applications, such as a database server, and may include software applications stored thereon configured to categorize, organize, and/or store data. In some embodiments, the calendar database 14 may be integrated into, or be an integral component of, the client database 12. Generally, the calendar database 14 may also include a processing element 32, a memory element 34, a communication element 36, and a software program 38, as shown in FIG. 3. For example, the calendar database 14 may utilize scheduling software as its software program 30, which may be configured for receiving, providing, modifying, and storing calendar data. The calendar database 14 may be a cloud database accessible via the communication network 22 or a local database at the physical location of the first service provider.

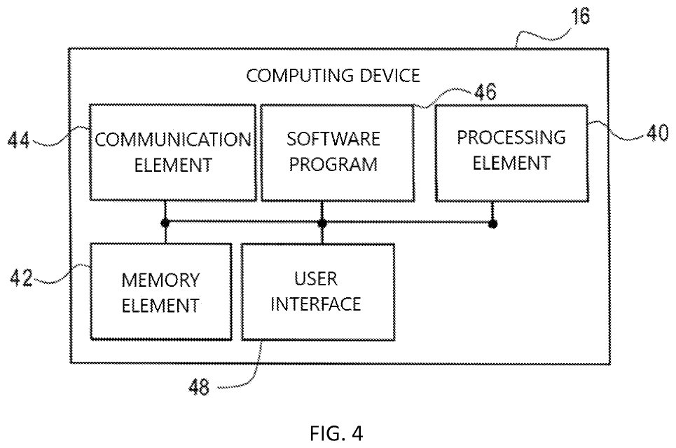

The first computing device 16 is connected to or in electronic communication with the client database 12, the calendar database 14, the second computing device 18, and the client device 20 via the communication network 22. Turning to FIG. 4, the first computing device 16 may comprise a tablet computer, a laptop computer, a desktop computer, a workstation computer, a smart phone, a smart watch, or the like. The first computing device 16 may comprise a single device or machine or a network of connected devices or machines. In addition, the first computing device 16 may include copiers, printers, routers and any other device that can connect to the communication network 22. The first computing device 16 may include a processing element 40, a memory element 42, a communication element 44, and a software application 46 configured with instructions for performing and/or enabling performance of at least some of the steps set forth herein. In an embodiment, the software application 46 comprises programs stored on computer-readable media of memory element 42. Still further, the first computing device 16 may include a user interface 48.

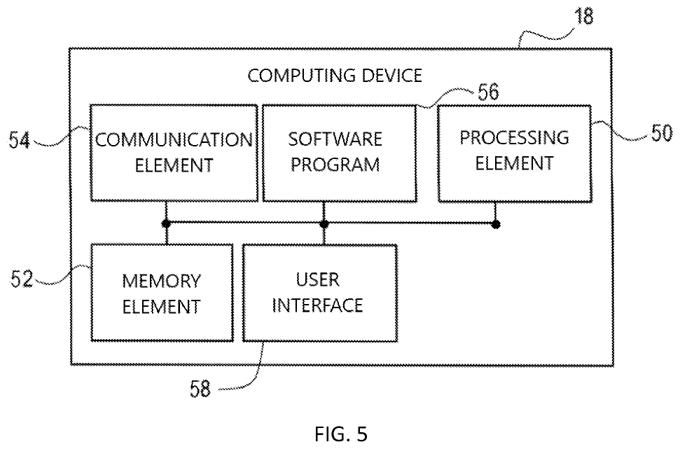

The second computing device 18 may also be connected to the communication network 22. Turning to FIG. 5, the second computing device 18 may comprise a tablet computer, a laptop computer, a desktop computer, a workstation computer, a smart phone, a smart watch, or the like. The second computing device 18 may comprise a single device or machine or a network of connected devices or machines. In addition, the second computing device 18 may include copiers, printers, routers and any other device that can connect to the communication network 22. The second computing device 18 may include a processing element 50, a memory element 52, a communication element 54, and a software application 56 configured with instructions for performing and/or enabling performance of at least some of the steps set forth herein. In an embodiment, the software application 56 comprises programs stored on computer-readable media of memory element 52. Still further, the first computing device 16 may include a user interface 58.

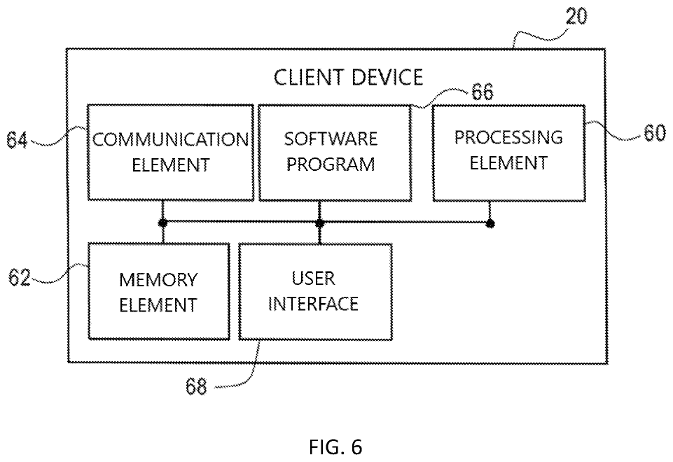

The client device 20 may also be connected to the communication network 22. Turning to FIG. 6, the client device 20 may comprise a tablet computer, a laptop computer, a desktop computer, a workstation computer, a smart phone, a smart watch, or the like. The client device 20 may include a processing element 60, a memory element 62, a communication element 64, a software application 66 configured with instructions for performing and/or enabling performance of at least some of the steps set forth herein, and a user interface 68. In an embodiment, the software application 56 comprises programs stored on computer-readable media of memory element 52.

The communication network 22 may be comprised of a plurality of networks including any internal networks that connect the client database 12, the calendar database 14, the first computing device 16, the second computing device 18, and the client device 20. A part of an internal network may be implemented via the first computing device 16. For instance, the first computing device 16 may execute a software program configured to provide a communication platform, which may comprise a trusted, secure internal network, messaging system, workspace system, database, or the like.

The communication network 22 generally allows communication between the client database 12, the calendar database 14, the first computing device 16, the second computing device 18, and the client device 20. The communication network 22 may include the Internet, cellular communication networks, local area networks, metro area networks, wide area networks, cloud networks, plain old telephone service (POTS) networks, and the like, or combinations thereof. The communication network 22 may be wired, wireless, or combinations thereof and may include components such as modems, gateways, switches, routers, hubs, access points, repeaters, towers, and the like. The client database 12, the calendar database 14, the first computing device 16, the second computing device 18, and the client device 20 may, for example, connect to the communication network 22 either through wires, such as electrical cables or fiber optic cables, or wirelessly, such as RF communication using wireless standards such as cellular 2G, 3G, 4G or 5G, Institute of Electrical and Electronics Engineers (IEEE) 802.11 standards such as WiFi, IEEE 802.16 standards such as WiMAX, Bluetooth™, or combinations thereof.

The processing elements 24, 32, 40, 50, 60 may include electronic hardware components such as processors. The processing elements 24, 32, 40, 50, 60 may include microprocessors (single-core and multi-core), microcontrollers, digital signal processors (DSPs), field-programmable gate arrays (FPGAs), analog and/or digital application-specific integrated circuits (ASICs), or the like, or combinations thereof. The processing elements 24, 32, 40, 50, 60 may generally execute, process, or run instructions, code, code segments, software, firmware, programs, applications, apps, processes, services, daemons, or the like. For instance, the processing elements 24, 32, 40, 50, 60 may respectively execute the software applications/program 30, 38, 46, 56, 66. The processing elements 24, 32, 40, 50, 60 may also include hardware components such as finite-state machines, sequential and combinational logic, and other electronic circuits that can perform the functions necessary for the operation of the current invention. The processing elements 24, 32, 40, 50, 60 may be in communication with the other electronic components through serial or parallel links that include universal busses, address busses, data busses, control lines, and the like.

The memory elements 26, 34, 42, 52, 62 may include electronic hardware data storage components such as read-only memory (ROM), programmable ROM, erasable programmable ROM, random-access memory (RAM) such as static RAM (SRAM) or dynamic RAM (DRAM), cache memory, hard disks, floppy disks, optical disks, flash memory, thumb drives, universal serial bus (USB) drives, or the like, or combinations thereof. In some embodiments, the memory elements 26, 34, 42, 52, 62 may be embedded in, or packaged in the same package as, the processing elements 24, 32, 40, 50, 60. The memory elements 26, 34, 42, 52, 62 may include, or may constitute, a “computer-readable medium.” The memory elements 26, 34, 42, 52, 62 may store the instructions, code, code segments, software, firmware, programs, applications, apps, services, daemons, or the like that are executed by the processing elements 24, 32, 40, 50, 60. In an embodiment, the memory elements 26, 34, 42, 52, 62 respectively store the software applications/program 30, 38, 46, 56, 66. The memory elements 26, 34, 42, 52, 62 may also store settings, data, documents, sound files, photographs, movies, images, databases, and the like.

The communication elements 28, 36, 44, 54, 64 generally allow communication between the client database 12, the calendar database 14, the first computing device 16, the second computing device 18, the client device 20, and the communication network 22. The communication elements 28, 36, 44, 54, 64 may include signal or data transmitting and receiving circuits, such as antennas, amplifiers, filters, mixers, oscillators, digital signal processors (DSPs), and the like. The communication elements 28, 36, 44, 54, 64 may establish communication wirelessly by utilizing radio frequency (RF) signals and/or data that comply with communication standards such as cellular 2G, 3G, 4G or 5G, Institute of Electrical and Electronics Engineers (IEEE) 802.11 standard such as WiFi, IEEE 802.16 standard such as WiMAX, Bluetooth™, or combinations thereof. In addition, the communication elements 28, 36, 44, 54, 64 may utilize communication standards such as ANT, ANT+, Bluetooth™ low energy (BLE), the industrial, scientific, and medical (ISM) band at 2.4 gigahertz (GHz), or the like. Alternatively, or in addition, the communication elements 28, 36, 44, 54, 64 may establish communication through connectors or couplers that receive metal conductor wires or cables, like Cat 6 or coax cable, which are compatible with networking technologies such as ethernet. In certain embodiments, the communication elements 28, 36, 44, 54, 64 may also couple with optical fiber cables. The communication elements 28, 36, 44, 54, 64 may respectively be in communication with the processing elements 24, 32, 40, 50, 60 and/or the memory elements 26, 34, 42, 52, 62.

The user interfaces 48, 58, 68 generally facilitate interactions between a user and one of the devices 16, 18, 20. The user interfaces 48, 58, 68 may comprise displays, speakers, or the like for providing information to the user and may comprise keyboards, touch screens, computer mouses, microphones, buttons, or the like for receiving input from a user.

The first computing device 16 may be configured to interact or exchange data with the client database 12, the calendar database 14, the second computing device 18, and/or the client device 20 to detect an opening event. The first computing device 16 may be configured to receive client data generated via the user interface 48. The client data may include an indication that a client wants to be notified of openings and corresponding preferences, such as a desired means for notifying the client, calendar data representing availability of the client, a preferred method of filling the opening (such as virtual or in-person), a preferred service provider, an amount of time that the client to arrive at the facility of the service provider (or other geographic data), or the like. The communication element 44 of the first computing device 16 may be configured to receive the client data from the client device 20 via the communication network 22. The processing element 40 of the first computing device 16 may be configured to store at least a portion of the received client data in the client database 12, the calendar database 14, and/or the memory element 42 of the first computing device 16.

The first computing device 16 may also be configured to authenticate the client device 20. The client device 20 may be configured to download the software program 66, which may instruct the user interface 68 to prompt the client for the relevant authentication and/or client data. In some embodiments, the software program 66 may comprise an internet browser that accesses a website associated with the first computing device 16. The client data may be inputted via the user interface 68 of the client device 20, and the software program 66 may instruct the communication element 64 to send the client data to the first computing device 16 via the communication network 22. The first computing device 16 may be configured to compare the received client data with pre-existing client data stored on the client database 12 and/or the memory element 42 of the first computing device 16. If the received client data is consistent with the pre-existing client data, then the first computing device 16 may then send, via the communication element 44, a notification of authentication to the client device 20 via the communication network 22. The software program 66 may instruct the processing element 60 to create and/or access an account associated with the client data and/or the client device 20. The client device 20 may be authenticated any number of ways without departing from the scope of the present invention.

As an example, relating to detecting an opening, the processing element 40 may be configured to periodically retrieve calendar data from the calendar database 14 and use the calendar data to detect an opening. The calendar data associated with a service provider may comprise one or more hour slots associated with a date. Each of the hour slots may be assigned to client data, such as client identification, or an indicator that the slot is filled. The service provider may increase her or his availability, such as by cancelling a slot indicated as “time off” or by increasing a number of available slots in a day. The software program 46 may instruct the processing element 40 to review the slots associated with the service provider and detect any opening in the calendar data associated with that service provider. Further, if the first computing device 16 receives, e.g., via the user interface 48, an indication of a cancelled appointment, a no-show, or the like, then software program 46 may instruct the processing element 40 to adjust the calendar data to open one of the hour slots. The software program 46 may instruct the processing element 40 to detect or flag the resulting opening. The software program 46 may instruct the processing element 40 to detect or flag an opening in the calendar data any number of ways without departing from the scope of the present invention. Moreover, the slots may be of any size (e.g., half-hour or the like) without departing from the scope of the present invention.

The first computing device 16 may be configured to automatically generate a list of clients to contact regarding the opening. In some embodiments, the software program 46 may instruct the processing element 40 to automatically request from the client database 12 client contact data associated with clients that match pre-established criteria. The criteria may comprise a positive indication of interest in being contacted to fill an opening, a pre-existing client account, a classification code (such as an ICD discussed above), calendar data indicating availability during the opening, preference settings indicating a willingness to participate in virtual appointments, client data indicating virtual appointments are consistent with the type of client (i.e., the client might be a patient needing an appointment does not require an in-person examination, blood work, or the like), a distance between client residences/work locations and the facility of the service provider, an amount of time the client indicated and/or that is automatically calculated by the first computing device 16 for transport to the facility of the service provider (in the case of an in-person session), an amount of time since the last session the client had with the service provider or a similar service provider, and/or other criteria. The software program 46 may instruct the processing element 40 to use the contact data of clients matching the criteria and the communication element 44 to automatically notify one or more of the clients (such as sending a message to corresponding client devices 20) of the opening via the communication network 22.

In some embodiments, the first computing device 16 may be configured to notify one or more service providers about the opening. For example, the software program 46 may instruct the processing element 40 to present a notification at the user interface 48 of the first computing device 16. As discussed previously, the first computing device 16 may comprise a network of devices. For example, the notification may be presented at a user interface 48 of a device associated with a nurse, doctor, receptionist, or the like, and the device may be a smart phone, pager, tablet, laptop, computer workstation, or the like. The software program 46 may instruct the user interface 48 to prompt the service provider to provide criteria for generating a list of one or more clients for contacting to fill the opening. The software program 46 may instruct the user interface 48 to prompt the service provider to set the criteria for contacting one or more of the clients. The software program 46 may also instruct the user interface 48 to prompt the service provider to select an order for the list of clients to be contacted. The order may be according to a selected priority variable, such as number of times a client has been notified previously, the number of times a client has rejected an opening, a distance between the client's residential/work address and the facility of the service provider, or the like. The software program 46 may instruct the user interface 48 to capture the selected criteria. The software program 46 may instruct the processing element 40 to pass the selected criteria to the client database 12 for retrieving contact data associated with clients consistent with the criteria.

In some embodiments, the first computing device 16 may be configured to contact the second computing device 18 to facilitate a joint appointment. For example, the software program 46 may instruct the user interface 48 to prompt the service provider to make a selection indicating a joint appointment is desired. In such embodiments, the joint appointment indication can be received without a detected opening. For example, the user interface 48 may receive instructions from the service provider to convert an existing appointment into a joint appointment. The software program 46 may instruct the processing element 40 to request, via the communication element 44 and the communication network 22, calendar data from the second computing device 18 and the client device 20. The request may include relevant client data, calendar data associated with the client, and/or calendar data associated with the first service provider. The first computing device 16 may receive, via the communication element 44, an indication of confirmation/acceptance of the joint appointment from the second computing device 18.

In some embodiments, the program 46 may instruct the processing element 40 to send a request for a range of calendar data of the second service provider, via the communication element 44 and the communication network 22, to the second computing device 18. The first computing device 16 may be configured to receive the range of calendar data of the second service provider, via the communication element 44. The program 46 may instruct the processing element 40 to determine a best time for a joint appointment. For example, the program 46 may instruct the processing element 40 to determine an opening in the calendar data of the second service provider that is shared by the calendar data associated with the first service provider. The program 46 may instruct the processing element 40 to compare the shared opening with the availability of one or more of the clients from the client data in the client database 12 to determine whether the shared opening falls within the availability of one or more of the clients.

The first computing device 16 may be configured to send notifications to one or more of the client devices 20. For example, the program 46 may instruct the processing element 40 to send a notification regarding the opening, via the communication element 44 and the communication network 22, to the one or more client devices 20 associated with one or more clients on the generated list. The notification may be sent on a rolling basis. Alternatively, the notification may be sent in a single batch, and the program 46 may instruct the processing element 44 to determine a first responsive client device 20. The program 46 may instruct the processing element 40 to also send the notification to one or more devices 20 associated with a third party that is associated with the client, such as for a joint appointment. The notification may be sent using an automated text messaging service, an automated call service, an automated email service, a notification on the program 66 of the client device 20, or the like. The notification may include calendar data associated with the opening, identification of the service provider (and in some embodiments, identification of the second service provider), information about the type of opening (i.e., length of the opening in terms of time, whether it is to be virtual or in person), an amount of time to respond to the notification, a link to a virtual appointment, and/or the like. The program 46 may instruct the processing element 40 to track an amount of time after sending a first notification to one of more of the client devices 20.

In some embodiments, the program 66 of the client device 20 may instruct the communication element 64 of the client device 20 to retrieve the notification via the communication network 22. The program 66 may instruct the processing element 60 to alert the client via the user interface 68, which may comprise a vibration, a sound, or the like. The program 66 may instruct the processing element 60 to display the notification via the user interface 68 and prompt the client to respond to the notification. The response may be received via the user interface 68, and the program 66 may instruct the communication element 64 to send the response via the communication element 64 and the communication network 22 to the first computing device 16. The response may comprise an indication of acceptance or rejection of the opening. The response may also comprise instructions to adjust preference data associated with the client, such as a change in availability, an indication of a desire to not receive such notifications, or the like. The program 66 may instruct the processing element 60 to also track the amount of time since receiving the notification from the first computing device 16.

If the user interface 68 of the client device 20 does not receive a response within a designated amount of time, the program 66 may instruct the processing element 60 to display a notification of the lapsed time via the user interface 68. Additionally or alternatively, the program 46 of the first computing device 16 may instruct the processing element 40 thereof to send a notification of the lapsed time to the client device 20 via the communication element 44 and the communication network 22, which may be received at the client device 20 and displayed via the user interface 68 of the client device 20.

The first computing device 16 may be configured to contact the next client on the list. For example, the program 46 of the first computing device 16 may instruct the processing element 40 to retrieve the response regarding the opening, via the communication element 44 and the communication network 22, from the one or more client devices 20. The program 46 of the first computing device 16 may instruct the processing element 40 to automatically send a notification to the next client on the list if the response is a rejection of the opening and/or the response time lapsed. The program 46 may also instruct the processing element 40 to record the response and/or lack of timely response in the client database 12 and/or the memory element 42 in association with that client. The program 46 may also instruct the processing element 40 to record any update in client preferences in the client database 12 and/or the memory element 42 in association with that client. This enables the tracking of responsive clients for list ordering purposes and/or adjusting client data to indicate that such clients are not open to receiving notifications about openings. The time requirement to respond encourages the rapid filling of a last-minute cancellation or no-show to minimize any lost revenue. Further, the ability to transition rapidly to a virtual appointment also reduces lost time and revenue. The first computing device 16 may be configured to contact the clients any number of ways without departing from the scope of the present invention.

The client device 20 may also receive and relay acceptance of the opening to the first computing device 16. If the user interface 68 of the client device 20 receives a response indicating acceptance of the appointment in the opening within the designated amount of time, the program 66 may instruct the communication element 64 to relay the acceptance to the first computing device 16. In some embodiments, the acceptance may be in the form of the client selecting a button representative of the link to the virtual meeting in the notification via the user interface 68. If the link was not included in the notification, the first computing device 16 may provide it later.

After acceptance, the client may be prompted to authenticate his or herself. For example, the program 66 may instruct the user interface 68 to request and receive authenticating data, such as account login credentials, client identification information, or the like. The program 66 may instruct the communication element 64 to send the authenticating data to the first computing device 16 and/or the client database 12 via the communication network 22. Those devices 16, 12 may determine that the authenticating data is consistent with their client data associated with that client and send an authentication confirmation notice to the client device 20. Any means or method of authenticating the client and/or client device 20 may be used without departing from the scope of the present invention.

Once the client has received the authentication confirmation notice, client data may be requested from the client in preparation for the appointment. For example, the program 66 may instruct the user interface 68 to request certain data from the client. In some embodiments, the program 66 may instruct the processing element 60 to receive the client data via the user interface 68 before progressing to a certain step in the program 66, or the program may instruct the processing element 60 to receive the client data at a later time, such as if the opening is more than five days away. The client data may include payment information, insurance information, medical information, and/or the like.

When the client has accepted, the first computing device 16 may be configured to fill the opening. The program 46 may instruct the processing element 40 to adjust the calendar data in the calendar database 14, such as by sending an indication that the opening is filled in association with the client that accepted.

The first computing device 16 may also be configured to notify the first service provider and, where applicable, the second service provider. The program 46 may instruct the processing element 40 to display a notification on the user interface 48. If the appointment filling the opening is an immediate/current virtual appointment, then the program 46 may instruct the processing element 40 to display on the user interface 48 a button representing a link for hosting a virtual appointment with the client. The processing element 40 may be configured to request (and in some embodiments, automatically request) client data associated with the client/client device 20 from the client database 12. The processing element 40 may be configured to display the client data on the user interface 48.

### Exemplary Method for Filling Open Appointments

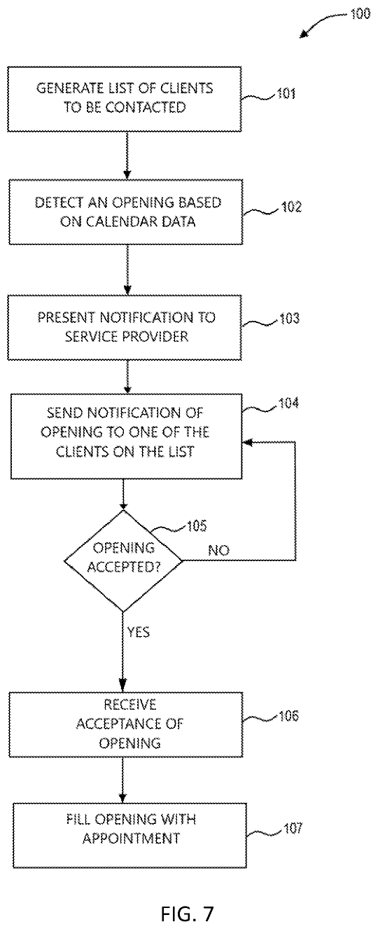

FIG. 7 depicts a flowchart including a listing of steps of an exemplary computer-implemented method 100 for filling an appointment. The steps may be performed in the order shown in FIG. 7, or they may be performed in a different order. Furthermore, some steps may be performed concurrently as opposed to sequentially. In addition, some steps may be optional.

The computer-implemented method 100 is described below, for ease of reference, as being executed by exemplary devices and components introduced with the embodiments illustrated in FIGS. 1-6. For example, the steps of the computer-implemented method 200 may be performed by one or more of the client database 12, the calendar database 14, the first computing device 16, the second computing device 18, the client device 20, and the communication network 22 through the utilization of processors, transceivers, hardware, software, firmware, or combinations thereof. However, a person having ordinary skill will appreciate that responsibility for all or some of such actions may be distributed differently among such devices or other computing devices without departing from the spirit of the present invention.

One or more computer-readable medium(s) may also be provided. The computer-readable medium(s) may include one or more executable programs stored thereon, wherein the program(s) instruct one or more processing elements to perform all or certain of the steps outlined herein. The program(s) stored on the computer-readable medium(s) may instruct the processing element(s) to perform additional, fewer, or alternative actions, including those discussed elsewhere herein.

Referring to step 101, a list of clients to be contacted may be generated. In some embodiments, a prompt may be displayed at a user interface of a first computing device, requesting definition, selection or other provision of criteria for generating a list of one or more clients for contacting to fill opening(s). The pre-defined and/or inputted criteria may comprise a positive indication of client interest in being contacted to fill an opening, a pre-existing client account, a classification code (such as an ICD discussed above and/or procedural codes such as Current Procedural Terminology (CPT) codes), calendar data indicating availability during the opening, preference settings indicating a willingness to participate in virtual appointments, client data indicating virtual appointments are consistent with the type of client (i.e., the client might be a patient needing an appointment does not require an in-person examination, blood work, or the like), a distance between client residences/work locations and the facility of the service provider, an amount of time the client indicated it would take to arrive at the facility of the service provider, and/or other criteria.

The prompt may also enable input defining a desired order in which the list of clients should be contacted. The order may be according to a selected priority variable, such as number of times a client has been notified previously, the number of times a client has rejected an opening, a distance between the client's residential/work address and the facility of the service provider, or the like. Once the criteria are established, the selected criteria may be passed to the client database for retrieving contact data associated with clients consistent with the criteria. In some embodiments, a list of client contact data associated with clients that match pre-established criteria may automatically be generated.

In an embodiment, the client database includes one or more ICD and/or CPT codes indicating medical services sought by one or more clients represented therein. The first computing device may automatically cross-reference one or more such codes against the type of opening in question (e.g., virtual or in-person), and may omit any clients associated with codes requiring a different type of opening. In one or more embodiments, the first computing device may require service provider input to make such an omission and/or may simply display any such perceived incompatibilities at the user interface of the first computing device, permitting the service provider to decide whether such an omission is necessary.

The client data may be pre-existing in the client database and/or generated via a client device. The client data may include an indication that a client wants to be notified of openings and corresponding preferences, such as a desired means for notifying the client, calendar data representing availability of the client, a preferred method of filling the opening (such as virtual or in-person), a preferred service provider, an amount of time that it will take the client to arrive at the facility of the service provider (or other geographic data), or the like. The client data may be received via a user interface of the first computing device. The client data may be stored at least partially in the client database, the calendar database, and/or the memory element of the first computing device.

Alternatively or additionally, the client data may be received from the client device via the communication network. In some embodiments, this step may include authenticating a client device and/or an account based on client data. A prompt may be displayed via a user interface of a client device to provide client data. The client data may be received via the user interface of the client device and sent to the first computing device via the communication network. The received client data may be compared, via the processing element of the first computing device, with pre-existing client data stored on the client database and/or the memory element of the first computing device. If the received client data is consistent with the pre-existing client data, then a notification of authentication may be sent to the client device via the communication network. If no matching, existing account is identified, an account associated with the client data and/or with the client device may be created.

In some embodiments, this step may comprise displaying a prompt to indicate a joint appointment is necessary. In such embodiments, the joint appointment indication can be received without a detected opening (described in more detail below). For example, instructions may be received via the user interface of the first computing device to convert an existing single-provider appointment and/or a single-patient appointment into a joint appointment. Calendar and contact data may be requested, via a communication element, from the second computing device, the client database, and/or the client device. The request may include relevant client data (such a preferred second service provider and/or a third party, such as a guardian or caretaker), calendar data associated with the client and/or the third party, and/or calendar data associated with the second service provider.

For example, a request for a range of calendar data of the second service provider may be sent, via the communication element and the communication network, to the second computing device associated with the second service provider. The range of calendar data of the second service provider may be received at the first computing device, via the communication element. A best time for a joint appointment may be determined based on the range of calendar data. For example, an opening in the calendar data of the second service provider that is shared by the calendar data associated with the first service provider may be determined, via the processing element of the first computing device, as described in more detail below. The shared opening may be compared with the availability of one or more of the clients from the client data in the client database to determine whether the shared opening falls within the availability of one or more of the clients.

In an embodiment, the first computing device may automatically compare one or more ICD and/or CPT codes indicating medical services sought by one or more clients in connection with identifying one or more second service providers for participation in a joint appointment. The first computing device may automatically cross-reference one or more such codes against the credentials and/or other qualifications of the potential second service providers, and may omit any potential second service providers lacking the qualifications necessary to provide such service(s). In one or more embodiments, the first computing device may require first service provider input to make such an omission and/or may simply display any such perceived incompatibilities at the user interface of the first computing device, permitting the first service provider to decide whether such an omission is necessary prior to transmission of notification(s) to one or more second service provider(s).

Referring to step 102, an opening may be detected based on calendar data. For example, the first computing device associated with the first service provider may interact with the client database, the calendar database, the second computing device associated with a second service provider, and/or the client device to detect an opening event. For example, the first computing device may be configured to detect, via a processing element thereof, an opening for the first service provider based on calendar data. The calendar data may be received from the calendar database. The calendar data may be associated with the first service provider and may comprise a number of hour slots associated with a date. Each of the hour slots may be assigned to client data, such as client identification, or may otherwise be associated with an indicator that the slot is filled. An opening may also be detected or flagged when the service provider increases her or his availability, such as cancelling a slot indicated as “time off” or the like or increasing a number of available slots in a day.

The slots associated with the service provider may be reviewed to detect or flag the opening in the calendar data associated with that service provider. As another example, if an indication of a cancelled appointment, a no-show, or the like, is received at the user interface of the first computing device, then calendar data may be adjusted to open one of the hour slots, which may be used to detect or flag the opening. However, an opening may be detected or flagged any number of ways without departing from the scope of the present invention.

Referring to step 103, a notification of the opening may be presented to the service provider. In some embodiments, a notification may be displayed at the user interface of the first computing device. For example, the notification may be presented at a user interface of a device associated with a nurse, a doctor, a receptionist, or the like, and the device may be a smart phone, pager, tablet, laptop, computer workstation, or the like.

Referring to step 104, a notification regarding the opening may be sent to one or more clients on the list. The notification regarding the opening may be sent, for example, via the communication element of the first computing device and the communication network, to the one or more client devices associated with one or more clients on the generated list. The notification may be sent to one or more devices associated with a third party that is associated with the client, such as a guardian or caretaker, for example as specified in the client database according to client inputs. The notification may be sent using an automated text messaging service, an automated call service, an automated email service, a notification on a program of the client device, or the like. The notification may include calendar data associated with the opening, identification of the service provider (and in some embodiments, the identification of the second service provider), information about the type of opening (i.e., length of the opening in terms of time, whether it is to be virtual or in person), an amount of time to respond to the notification, a link to a virtual appointment, and/or the like. This step may include tracking an amount of time after sending a first notification to one of more of the client devices. As discussed above, for a shared opening, the notification or a corresponding notification may also be sent to a second computing device associated with the second service provider. In one or more embodiments, one or more acceptances of opening(s) may be received from the second service provider prior to and/or as a condition of sending the notification to the client device(s).

The notification may be received at the client device. The client may be alerted via the client device, such as a notification displayed via the user interface, a vibration, a sound, and/or the like. The notification may comprise a prompt for the client. A response from the client may be received via the user interface of the client device, and the response may be sent via the communication element of the client device over the communication network to the first computing device. The response may comprise an indication of acceptance or rejection of the opening. The response may also comprise instructions to adjust preference data associated with the client, such as a change in availability, an indication of a desire to not receive such notifications, or the like.

Referring to step 105, a determination of whether the client has accepted and/or responded in time is made. If a response is not received, for example via the user interface of the client device, within a designated amount of time, a notification of the lapsed time may be displayed on the client device. Additionally or alternatively, a notification of the lapsed time may be sent to the client device from the first computing device. A notification of the opening may be sent to the next client on the list if the response is a rejection of the opening and/or the response time lapsed. In one or more embodiments, several clients may be notified in tandem, with the first to respond affirmatively being awarded the opening and the slower respondees receiving an automated notification that the opening has closed. Response and/or lack of a timely response may be recorded in the client database and/or the memory element of the first computing device in association with each client. Any update in client preferences included in the response may be recorded in the client database and/or the memory element of the first computing device in association with that client.

Referring to step 106, a notification of acceptance is received. For example, the acceptance of the opening may be received via the user interface of the client device and sent to the first computing device. This step may include receiving a selection of a button representative of a link to a virtual meeting in the notification via the user interface of the client device. In some embodiments, this step may include authenticating the client and/or the client device. For example, authenticating data, such as account login credentials, client identification information, or the like may be requested and received. The authenticating data may be sent to the first computing device and/or the client database via the communication network. The authenticating data may be compared with existing client data to determine whether the authenticating data is consistent with the client data associated with that client. An authentication confirmation notice may be to the client device if the authenticating data is consistent with pre-stored client data. Any means or method of authenticating the client and/or client device may be used without departing from the scope of the present invention. This step may include requesting and receiving additional client data from the client in preparation for the appointment. For example, the additional client data may include payment information, insurance information, medical/health information, and/or the like.

Referring to step 107, the opening is filled. When the client has accepted, the calendar data may be adjusted to indicate that the opening has been filled. This may include sending the calendar data to the calendar database and/or assigning the slot of the opening to client data/identification associated with the accepting client. This step may include sending a notification to the first computing device and/or the second computing device and a device associated with a third party (such as when there is a joint appointment). If the appointment filling the opening is an immediate/current virtual appointment, then a button representing a link for hosting a virtual appointment with the client may be presented on one or more of the user interfaces of the computing devices. Client data associated with the client/client device may be received from the client database and displayed on one or more of the computing devices and/or the client device.

The method may include additional, less, or alternate steps and/or device(s), including those discussed elsewhere herein. For example, the method may include a step for prompting the service provider to provide updated client data and storing the updated client data in the client database. The method may also include prompting the client to provide availability for a follow-up appointment, such as an in-person appointment, and converting the availability into calendar data for storing in the calendar database and/or the client database.

### Additional Considerations

In this description, references to “an embodiment”, “an embodiment”, or “embodiments” mean that the feature or features being referred to are included in at least an embodiment of the technology. Separate references to “an embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in an embodiment may also be included in other embodiments, but is not necessarily included. Thus, the current technology can include a variety of combinations and/or integrations of the embodiments described herein.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as computer hardware that operates to perform certain operations as described herein.

In various embodiments, computer hardware, such as a processing element, may be implemented as special purpose or as general purpose. For example, the processing element may comprise dedicated circuitry or logic that is permanently configured, such as an application-specific integrated circuit (ASIC), or indefinitely configured, such as an FPGA, to perform certain operations. The processing element may also comprise programmable logic or circuitry (e.g., as encompassed within a general-purpose processor or other programmable processor) that is temporarily configured by software to perform certain operations. It will be appreciated that the decision to implement the processing element as special purpose, in dedicated and permanently configured circuitry, or as general purpose (e.g., configured by software) may be driven by cost and time considerations.

Accordingly, the term “processing element” or equivalents should be understood to encompass a tangible entity, be that an entity that is physically constructed, permanently configured (e.g., hardwired), or temporarily configured (e.g., programmed) to operate in a certain manner or to perform certain operations described herein. Considering embodiments in which the processing element is temporarily configured (e.g., programmed), each of the processing elements need not be configured or instantiated at any one instance in time. For example, where the processing element comprises a general-purpose processor configured using software, the general-purpose processor may be configured as respective different processing elements at different times. Software may accordingly configure the processing element to constitute a particular hardware configuration at one instance of time and to constitute a different hardware configuration at a different instance of time.

Computer hardware components, such as transceiver elements, memory elements, processing elements, and the like, may provide information to, and receive information from, other computer hardware components. Accordingly, the described computer hardware components may be regarded as being communicatively coupled. Where multiple of such computer hardware components exist contemporaneously, communications may be achieved through signal transmission (e.g., over appropriate circuits and buses) that connect the computer hardware components. In embodiments in which multiple computer hardware components are configured or instantiated at different times, communications between such computer hardware components may be achieved, for example, through the storage and retrieval of information in memory structures to which the multiple computer hardware components have access. For example, one computer hardware component may perform an operation and store the output of that operation in a memory device to which it is communicatively coupled. A further computer hardware component may then, at a later time, access the memory device to retrieve and process the stored output. Computer hardware components may also initiate communications with input or output devices, and may operate on a resource (e.g., a collection of information).

The various operations of example methods described herein may be performed, at least partially, by one or more processing elements that are temporarily configured (e.g., by software) or permanently configured to perform the relevant operations. Whether temporarily or permanently configured, such processing elements may constitute processing element-implemented modules that operate to perform one or more operations or functions. The modules referred to herein may, in some example embodiments, comprise processing element-implemented modules.

Similarly, the methods or routines described herein may be at least partially processing element-implemented. For example, at least some of the operations of a method may be performed by one or more processing elements or processing element-implemented hardware modules. The performance of certain of the operations may be distributed among the one or more processing elements, not only residing within a single machine, but deployed across a number of machines. In some example embodiments, the processing elements may be located in a single location (e.g., within a home environment, an office environment or as a server farm), while in other embodiments the processing elements may be distributed across a number of locations.

Unless specifically stated otherwise, discussions herein using words such as “processing,” “computing,” “calculating,” “determining,” “presenting,” “displaying,” or the like may refer to actions or processes of a machine (e.g., a computer with a processing element and other computer hardware components) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The patent claims at the end of this patent application are not intended to be construed under 35 U.S.C. § 112(f) unless traditional means-plus-function language is expressly recited, such as “means for” or “step for” language being explicitly recited in the claim(s).

Although the invention has been described with reference to the embodiments illustrated in the attached drawing figures, it is noted that equivalents may be employed and substitutions made herein without departing from the scope of the invention as recited in the claims.

Having thus described various embodiments of the invention, what is claimed as new and desired to be protected by Letters Patent includes the following:

1. A computer-implemented method for filling open appointments comprising:
determining, via one or more processors, a selected classification code of one or more classification codes associated with one or more clients in a client database associated with a service provider;
detecting, via the one or more processors, an event indicative of a change in calendar data, the change in calendar data comprising an opening of a time slot in a schedule of the service provider;
determining, via the one or more processors, that the opening should be filled;
generating, via the one or more processors, a list of one or more clients associated with the selected classification code from the client database; and
sending, via the one or more processors, a signal representative of a notification regarding the opening of the time slot to one or more devices associated with the one or more clients associated with the selected classification code.

2. The computer-implemented method of claim 1, further comprising
determining, via the one or more processors, a desired response time for the notification regarding the opening of the time slot; and
counting, via the one or more processors, a first amount of time after the signal representative of the notification regarding the opening was sent to a first device associated with a first client of the one or more clients associated with the selected classification code.

3. The computer-implemented method of claim 2, further comprising
determining, via the one or more processors, that the desired response time has expired based on the first amount; and
sending, via the one or more processors, a signal representative of a notification regarding the opening of the time slot to a second device associated with a second client of the one or more clients associated with the selected classification code.

4. The computer-implemented method of claim 2, further comprising
receiving, via the one or more processors, a signal representative of an acceptance of the opening of the time slot from the first device associated with the first client within the desired response time; and
sending, via the one or more processors, a signal representative of a confirmation of the acceptance, the confirmation comprising a link to a telehealth encounter.

5. The computer-implemented method of claim 1, further comprising
receiving, via the one or more processors, a signal representative of an acceptance of the time slot from the one or more devices associated with the one or more clients; and
modifying, via the one or more processors, the calendar data to indicate that the opening of the time slot has been filled.

6. The computer-implemented method of claim 1, further comprising
receiving, via the one or more processors, first client data;
entering, via the one or more processors, the first client data into a client database;
receiving, via the one or more processors, a registration notification and corresponding second client data from a client device; and
determining, via the one or more processors, that the first client data is consistent with the second client data.

7. The computer-implemented method of claim 1, further comprising
determining, via the one or more processors, a priority variable associated with each of the one or more clients in the client database; and
ordering, via one or more processors, the list according to the priority variable.

8. The computer-implemented method of claim 1, wherein the classification codes comprise international classification of diseases codes.

9. A system for filling open appointments, the system comprising:
a client database configured to store electronic medical records for clients, the electronic medical records including client identification data, classification codes associated with client identification data, and contact data associated with client identification data;
a calendar database configured to store calendar data associated with a service provider, the calendar data comprising time slots assigned to clients representative of appointments;
a first computing device in communication with the client database and the calendar database and configured to
detect an event indicative of a change in the calendar data, the change in the calendar data comprising an opening of a time slot of the service provider;
generate a list of one or more classification codes associated with one or more clients in the client database;
receive a signal representative of a selected classification code;
generate a list of one or more clients associated with the selected classification code from the client database; and
send a signal representative of a notification regarding the opening of the time slot using the contact data associated with the one or more clients associated with the selected classification code.

10. The system of claim 9, wherein the first computing device is configured to
receive a signal representative of a desired response time for the notification regarding the opening of the time slot; and
count a first amount of time after the signal representative of the notification regarding the opening was sent.

11. The system of claim 10, wherein the first computing device is configured to
determine that the desired response time has expired based on the first amount; and
send a signal representative of a notification regarding the opening of the time slot using the contact data associated with a second client of the one or more clients associated with the selected classification code.

12. The system of claim 11, wherein the client database is configured to store one or more priority variables associated with one or more of the clients, wherein the first computing device is configured to order the list according to the one or more priority variables associated with the one or more clients associated with the selected classification code.

13. The system of claim 12, wherein the one or more priority variables comprise a number of times the one or more clients rejected the notification regarding the opening.

14. The system of claim 12, wherein the one or more priority variables comprise a number of days since the one or more clients had an appointment with the service provider.

15. The system of claim 9, wherein the service provider is a first type of service provider, the first computing device being further configured to
receive a signal representative of a request for a joint appointment with one of the clients and a second type of service provider;
send a signal representative of a request for a joint appointment session for the opening of the time slot of the first service provider to a second computing device associated with the second type of service provider;
receive a signal representative of acceptance of the request for the joint appointment from the second computing device;
receive a signal representative of acceptance of the notification regarding the opening of the time slot from a device associated with the one or more clients associated with the selected classification code; and
send a signal representative of a confirmation of the acceptance to the second computing device and the device associated with the one or more clients associated with the selected classification code, the confirmation comprising a link to a telehealth encounter connection.

16. The system of claim 9, wherein the client database is configured to store calendar data regarding availability to receive notifications associated with the client identification data.

17. A computer-implemented method for filling open appointments comprising:
receiving, via one or more processors, a signal representative of a user input comprising a request for a joint appointment with a client and a second type of service provider;
sending, via the one or more processors, a signal representative of a request for calendar data associated with the first type of service provider and calendar data associated with the second type of service provider to a server;
receiving, via the one or more processors, a signal representative of the calendar data associated with the first type of service provider and calendar data associated with the second type of service provider from the server;
determining, via the one or more processors, a shared available time slot for the first type of service provider and the second type of service provider based on the calendar data associated with the first type of service provider and calendar data associated with the second type of service provider; and
sending, via one or more processors, a signal representative of a notification of the shared available time slot to a device associated with the client.

18. The computer-implemented method of claim 17, further comprising
receiving, via the one or more processors, a signal representative of an acceptance of the shared available time slot from the device associated with the client; and
sending, via the one or more processors, a signal representative of a request to assign the client to the shared available time slot of the first type of service provider and the second type of service provider to the server.

19. The computer-implemented method of claim 17, wherein the server is at least one of a server of first type of service provider, a server of the second type of service provider, an intermediary server in communication with an EMR database, or an EMR server in communication with the EMR database.

20. The computer-implemented method of claim 17, further comprising
sending, via the one or more processors, a signal representative of a request for third-party data associated with the client to the server;
receiving, via the one or more processors, a signal representative of the third-party data associated with the client; and
sending, via the one or more processors, a signal representative of a notification of the shared available time slot to a device associated with a third party.
